(19) United States
(12) Patent Application Publication
(10) Pub. No.: US 2016/0339074 A1
(43) Pub. Date: Nov. 24, 2016

(54) PHARMACEUTICAL COMPOSITION OF SELECTIVE HCV NS3/4A INHIBITORS

(71) Applicant: MERCK SHARP & DOHME CORP., Rahway, NJ (US)

(72) Inventors: Melanie J. Marota, Mount Laurel, NJ (US); Craig McKelvey, Ambler, PA (US); Nicholas Birringer, San Mateo, CA (US); Jesse Kuiper, North Wales, PA (US); Paul A. Harmon, Audubon, PA (US); Adam J. Socia, Blue Bell, PA (US); Patrick Jules Marsac, Lexington, KY (US); Stephen L. Conway, Jenkintown, PA (US)

(73) Assignee: Merck Sharp & Dohme Corp., Rahway, NJ (US)

(21) Appl. No.: 15/116,820

(22) PCT Filed: Feb. 3, 2015
(86) PCT No.: PCT/US2015/014195

§ 371 Date: Aug. 4, 2016

Related U.S. Application Data
(60) Provisional application No. 61/936,019, filed on Feb. 5, 2014.

(51) Int. Cl. A61K 38/06; A61K 9/16

(52) U.S. Cl. CPC A61K 38/06; A61K 9/1652; A61K 9/1635; A61K 9/1611; A61K 9/1641; A61K 9/1617

(57) ABSTRACT

The present invention is directed to compositions comprising the HCV NS3/4A inhibitor, (1aR,5S,8S,10R,22aR)-5-tert-butyl-N-{(1R,2S)-1-[(cyclopropylsulfonyl)carbamoyl]-2-ethenylcyclopro-pyl}-14-methoxy-3,6-dioxo-1,1a,3,4,5,6,9,10,18,19,20,21,22,22a-tetradecahydro-8H-7,10-methanocyclopropa[18,19][1,10,3,6]dioxa-diazacyclononadecino[11,12-b]quinoxaline-8-carboxamide, or a pharmaceutically acceptable salt thererof, an oral absorption enhancing polymer, and, optionally, a surfactant. The present invention is also directed to solid dispersions and pharmaceutical compositions containing or made from these compositions, and the methods for making these solid dispersions and pharmaceutical compositions.

### FIELD OF THE INVENTION

The present invention is directed to compositions comprising the HCV NS3/4A inhibitor, (1aR,5S,8S,10R,22aR)-5-tert-butyl-N-{(1R,2S)-1-[(cyclopropylsulfonyl)carbamoyl]-2-ethenylcyclopropyl}-14-methoxy-3,6-dioxo-1,1a,3,4,5,6,9,10,18,19,20,21,22,22a-tetradecahydro-8H-7,10-methanocyclopropa[18,19][1,10,3,6]dioxadiazacyclononadecino[11,12-b]quinoxaline-8-carboxamide, or a pharmaceutically acceptable salt thererof, an absorption enhancing polymer, and, optionally, a surfactant. The present invention is also directed to solid dispersions and pharmaceutical compositions containing or made from these compositions, and the methods for making these solid dispersions and pharmaceutical compositions.

### BACKGROUND OF THE INVENTION

The selective HCV NS3/4A inhibitor, (1aR,5S,8S,10R,22aR)-5-tert-butyl-N-{(1R,2S)-1-[(cyclopropylsulfonyl)carbamoyl]-2-ethenylcyclopropyl}-14-methoxy-3,6-dioxo-1,1a,3,4,5,6,9,10,18,19,20,21,22,22a-tetradecahydro-8H-7,10-methanocyclopropa[18,19][1,10,3,6]dioxadiazacyclononadecino[11,12-b]quinoxaline-8-carboxamide is shown as Compound 1

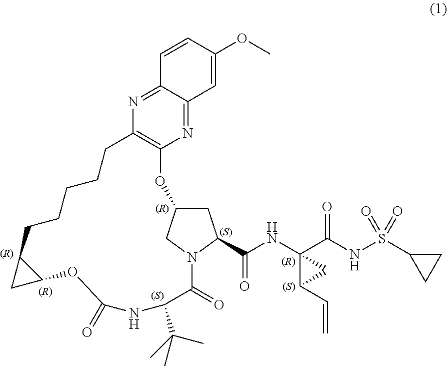

and is described in U.S. Pat. No. 7,973,040. Compound 1 is a moderately lipophilic compound (log D˜3 at pH=7) with a low crystallization tendency (TM/TG ratio of 1.12 based on the most stable crystalline phase known) and a very low aqueous solubility (<50 μg/ml).

The use of solid dispersions, and, particularly, solid solutions, to promote the oral absorption of poorly water soluble active pharmaceutical ingredients (APIs) is known. See, e.g, Ford, Pharm Acta Helv, 1986, 61:69-88. Solid dispersions are compositions in which APIs are dispersed into excipients. Solid solutions, defined as solid dispersions in which the active pharmaceutical ingredient forms a homogeneous or nearly homogeneous glass when dispersed into the excipient matrix, are of particular interest in the oral delivery of poorly water soluble compounds. It is believed that these solid solutions improve the absorption of orally administered APIs by improving the wetting properties of the API, causing transient supersaturation of the API with respect to a lower energy phase (e.g., crystalline API), or both. In general, solid solutions are believed to enable drug absorption by enhancing the dissolution rate and/or its extent.

Despite their growing use, the design of solid solution formulations to effectively promote oral drug absorption remains largely a matter of trial and error. Successful formulation of lipophilic compounds as solid dispersions to promote oral absorption may benefit from a strong interaction between API and polymer. This has led to interest in partially water soluble polymers with amphiphilic properties like hydroxypropyl methylcellulose acetate succinate (HPMCAS), especially when the process used to create the solid dispersion is spray drying. See Friesen et al., Mol. Pharm., 2008, 5:1003-1019. While this approach has been successful for many drug candidates, compounds with high melting points (or high ratios of melting point to glass transition temperature) and/or particularly lipophilic compounds (e.g., those with high log P values) are especially problematic to successfully formulate as solid solutions. Friesen et al. suggest successful formulations of compounds with these properties will likely be limited to relatively dilute concentrations of API in the solid dispersion. See Friesen et al., Mol. Pharm., 2008, 5:1003-1019. Friesen et al. in testing a select group of compounds found that compounds exhibiting relatively low Tm/Tg ratios (<1.25) and low to moderate log P values (less than about 6) were able to be successfully formulated as spray-dried dispersions with high drug loading (e.g., 50 wt % or more) while maintaining acceptable physical stability and rapid dissolution rates.

U.S. Pat. No. 4,801,460 describes solid dispersions comprising a poorly soluble drug and at least one copolymer of vinyl pyrrolidone and vinyl acetate.

The design of formulations of Compound 1 that provide effective absorption following oral administration is useful to reduce pill burden (e.g., the number of tablets administered) and regimen complexity (e.g., eliminating the need to administer with food). Formulations with this type of enhanced absorption will ultimately improve compliance and, therefore, efficacy.

### SUMMARY OF THE INVENTION

The present invention is directed to compositions of (1aR,5S,8S,10R,22aR)-5-tert-butyl-N-{(1R,2S)-1-[(cyclopropylsulfonyl)carbamoyl]-2-ethenylcyclopropyl}-14-methoxy-3,6-dioxo-1,1a,3,4,5,6,9,10,18,19,20,21,22,22a-tetradecahydro-8H-7,10-methanocyclopropa[18,19][1,10,3,6]dioxadiazacyclononadecino[11,12-b]quinoxaline-8-carboxamide, or a pharmaceutically acceptable salt thereof, formulated into solid dispersions and pharmaceutical compositions. In certain embodiments, the compositions described herein are believed to afford high dissolution rates and more complete absorption.

Thus, in one embodiment, the present invention provides a composition comprising: a) Compound 1, or a pharmaceutically acceptable salt thereof, in a concentration between about 0.1% to about 40% w/w; and b) an absorption enhancing polymer in a concentration between about 60% and about 99.9% w/w. In certain aspects of this embodiment, the concentration of Compound 1 in the composition is between about 5% and about 35% w/w. In another aspect of this embodiment, the concentration of Compound 1 is between about 10% to about 30% w/w. In an aspect of this embodiment, the composition is in the form of a particle.

In one embodiment of the invention, the absorption enhancing polymer is a cellulosic polymer. Examples of cellulosic polymers include HPMC (hydroxypropylmethyl cellulose), HPMCAS (hydroxypropylmethylcellulose acetate succinate) or HPMCP (hydroxypropylmethylcellulose phthalate). In one embodiment, the absorption enhancing polymer is a cellulosic polymer selected from HPMC, HPMCAS or HPMCP.

In another embodiment, the absorption enhancing polymer is a copolymer of vinyl pyrrolidone and vinyl acetate. The copolymer of vinyl pyrrolidone and vinyl acetate can be copovidone.

In another embodiment, the composition of the invention further comprises a surfactant in a concentration from about 2% to about 15%. The surfactant can be selected from sodium lauryl sulfate (SLS), D-α-tocopheryl polyethylene glycol 1000 succinate (vitamin E TPGS), or nonionic ethoxylated alcohols like polysorbate or poloxamer. In one embodiment, the surfactant is present at a concentration from about 5% to about 10% w/w and is selected from sodium lauryl sulfate (SLS) and D-α-tocopheryl polyethylene glycol 1000 succinate (vitamin E TPGS).

Another embodiment of the invention provides a solid dispersion comprising particles of the compositions described above. In certain aspects of this embodiment, the solid dispersion is formed by spray drying or extruding the compositions of the invention. In one aspect, the dispersion comprises particles wherein the surfactant is SLS and the dispersion is formed by spray drying using a mixed solvent system comprising a volatile solvent and a non-volatile solvent. In particular embodiments, the volatile solvent is selected from ethanol, methanol or acetone, while the non-volatile solvent is water. In specific embodiments, the mixed solvent system can be acetone:water (90:10). In select embodiments, the solid dispersion may be further formulated into a blended material comprising the solid dispersion, a salt, e.g., selected from NaCl, KCl, CaCl₂ or combinations thereof; and a disintegrant, e.g., selected from croscarmellose sodium, sodium starch glycolate or crospovidone. Thus, an embodiment of the invention includes a blended material comprising a solid dispersion of Compound 1 as described above, a salt selected from NaCl, KCl, CaCl₂ and combinations thereof, and a disintegrant selected from croscarmellose sodium, sodium starch glycolate and crospovidone. In one embodiment, the disintegrant is croscarmellose sodium. In another embodiment, the disintegrant is croscarmellose sodium and the salt is selected from NaCl or KCl.

Another embodiment of the invention provides a pharmaceutical formulation comprising a solid dispersion of the invention; a salt, e.g., selected from NaCl, KCl, CaCl₂ or a combination thereof; and a disintegrant, e.g., selected from croscarmellose sodium, sodium starch glycolate or crospovidone. In one embodiment, the disintegrant is croscarmellose sodium. In certain aspects of this embodiment, the disintegrant is present at a concentration of about 5-20% w/w. In another aspect of this embodiment, the disintegrant is present at a concentration of about 5-10% w/w. In another embodiment, the disintegrant is croscarmellose sodium and the salt is selected from NaCl or KCl. In certain aspects of this embodiment, the solid dispersion is present at a concentration of about 5-70% w/w or about 20-50% w/w of the pharmaceutical formulation.

In certain embodiments, the composition, blended material or pharmaceutical formulation further comprises a diluent system (e.g., mannitol, microcrystalline cellulose, calcium carbonate, sodium carbonate, lactose, calcium phosphate or sodium phosphate), lubricant (e.g., magnesium stearate, sodium stearyl fumarate, stearic acid or talc) and glidant (e.g., colloidal silicon dioxide), at concentrations of about 0-70% w/w, about 0.25-3.0% w/w, about 0-3.0% w/w, respectively, or at about 20-60% w/w, about 0.5-2.0%, and about 0-1.0% w/w, respectively.

In one aspect, the pharmaceutical formulation comprises about 10% w/w Compound 1, about 20-25% w/w polyvinylpyrrolidone/vinyl acetate copolymer, about 1-2% w/w sodium lauryl sulfate, about 45% w/w mannitol, about 10% w/w croscarmellose sodium, about 10% w/w sodium chloride, about 1.5% w/w magnesium stearate, and about 0.25% w/w colloidal silicon dioxide for a combined tablet weight that can vary from 100 mg to 2000 mg, 200 to 1500 mg, and 500 mg to 1000 mg. In one embodiment, the combined tablet weight is about 1000 mg. The pharmaceutical formulation may be formulated into an oral dosage form such as a capsule or tablet.

Another embodiment of the invention provides a process for preparing a solid pharmaceutical composition comprising the steps of: a) dissolving the compositions described above in a solvent system comprising a volatile solvent; b) spray-drying the dissolved composition to form particles; and c) compressing the particles into a tablet or filling them into a capsule. In an aspect of this embodiment, the process comprises the steps of: a) dissolving the compositions of the invention in a solvent system comprising a volatile solvent; b) spray-drying the dissolved composition to form particles; c) blending the particles with one or more of a diluent, disintegrant, salt, lubricant, and glidant; d) subjecting the blended particles to roller compaction; e) adding a lubricant; and f) compressing the particles into a tablet or filling them into a capsule. The volatile solvent can be selected from ethanol, methanol or acetone. In certain embodiments, the solvent system further comprises a non-volatile solvent, which can be water. Such a mixed solvent system can be acetone:water (90:10).

### BRIEF DESCRIPTION OF THE DRAWINGS

FIG. 1: Production Process for Spray Dried Compound 1 Intermediate 1

FIG. 2: Production Process for Formulation 4

FIG. 3: Production Process for Spray Dried Compound 1 Intermediate 2

FIG. 4: Production Process for Formulation 10

FIG. 5: Production Process for Spray Dried Compound 1 Intermediate 3

FIG. 6: Production Process for Formulation 11

FIG. 7: Production Process for Spray Dried Compound 1 Intermediate 4

FIG. 8: Production Process for Formulation 12

### DETAILED DESCRIPTION OF THE INVENTION

The present invention is based on the recognition that formulations of Compound 1 at a drug loading less than or equal to 40% in combination with absorption enhancing polymers are important for enabling absorption of the compound. Compound 1 is a moderately lipophilic compound (log D-3 at pH=7) with a low crystallization tendency (TM/TG ratio of 1.12 based on the most stable crystalline phase known) and a very low aqueous solubility (<50 μg/ml). Even in its amorphous state, the apparent solubility of neat amorphous Compound 1 in simulated gastric fluid and simulated fasted state intestinal fluid is <3 μg/mL and 50 μg/mL, respectively, after 2 hours of equilibration. Solid dispersions of Compound 1 in which the concentration of Compound 1 is greater than 50% by weight do not provide sufficient absorption of Compound 1 when delivered orally.

As demonstrated by the Examples, the oral absorption of Compound 1 when formulated as a solid solution together with absorption enhancing polymers, such as HPMCAS and copovidone, optionally together with surfactants including sodium lauryl sulfate (SLS) and vitamin E TPGS, is superior to formulations based on undispersed amorphous Compound 1 or crystalline Compound 1.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±0%, ±1%, ±2%, ±3%, ±4%, ±5%, ±10%, ±15%, and ±20% and their numerical equivalents.

The term “effective amount” indicates a sufficient amount to exert a therapeutic or prophylactic effect. For a patient infected with HCV, an effective amount is sufficient to achieve one or more of the following effects: reduce the ability of HCV to replicate, reduce HCV load, and increase viral clearance. For a patient not infected with HCV, an effective amount is sufficient to achieve one or more of the following: a reduced susceptibility to HCV infection, and a reduced ability of the infecting virus to establish persistent infection for chronic disease.

The term “subject” (alternatively referred to herein as “patient”) as used herein refers to an animal, preferably a mammal, most preferably a human, who has been the object of treatment, observation or experiment.

Compound 1 may be formulated using pharmaceutically acceptable salts. The term “pharmaceutically acceptable salt” refers to a salt of the parent compound that has activity and that is not biologically or otherwise undesirable (e.g., is neither toxic nor otherwise deleterious to the recipient thereof). Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Examples of salts and salt forms include those described in International Patent Application Publication No. WO2013/028465. Methods for making Compound 1, and pharmaceutically acceptable salts thereof, are described in International Patent Application Publication Nos. WO2013/028471 and WO2013/028470. A particularly preferred salt form is the crystalline potassium salt form of Compound 1 described therein. Crystal forms of Compound 1 are described in International Patent Application Publication No. WO2013/028465. In a specific embodiment, Compound 1 is in the form of a crystalline potassium salt characterized by an X-ray powder diffraction pattern obtained using copper Kₐ radiation which comprises 2Θ values in degrees of about 18.2, 8.9, and 20.3.

The compositions of the invention comprise an absorption enhancing polymer. Absorption enhancing polymers are soluble in water at a pH relevant to oral drug absorption (e.g., pH 1-7). Absorption enhancement can be reflective of an increase in the area under the plasma concentration profile (AUC) following oral dosing of a formulation. Dispersing Compound 1 in an absorption enhancing polymer results in superior absorption when compared with formulations containing undispersed amorphous Compound 1 (not in a solid solution). Evaluation of absorption enhancement is ideally conducted in humans, however, animal models may be indicative of likely absorption enhancement in humans.

In different embodiments, formulations based on molecular dispersions of Compound 1 in absorption enhancing polymers increase AUC by at least 20%, at least 30%, at least 40% or at least 50% relative to standard oral dosage formulations, e.g., capsules or tablets, based on undispersed amorphous (not molecularly dispersed) or neat crystalline phases of Compound 1. While absorption enhancing polymers may enable greater extent or duration of supersaturation of Compound 1 relative to a neat amorphous control, this type of increase in apparent solubility unexpectedly is not necessary to provide absorption enhancement. For example, concentration enhancing polymers may be important for enhancing the exposure of water insoluble compounds (See, e.g., U.S. Pat. No. 6,763,607). For Compound 1, concentration enhancement (e.g., supersaturation relative to a stable crystalline phase of Compound 1) is insufficient to demonstrate absorption enhancement, as shown in Example 2.

In an embodiment of the present invention, the absorption enhancing polymer is a vinyl pyrrolidinone/vinyl acetate copolymer. In one embodiment, the absorption enhancing polymer is copovidone, a copolymer of 1-vinyl-2-pyrrolidone and vinyl acetate in the mass proportion of 3:2. Other useful copolymers contain vinyl pyrrolidone and vinyl acetate in ratios of, for example, 90:10, 80:20, 70:30, and 50:50. The amount of vinyl pyrrolidone can range from about 40% up to about 99.9%, and the amount of vinyl acetate can range from about 0.1% up to about 60%. Other vinyl polymers and copolymers having substituents that are hydroxy, alkyl, acyloxy, or cyclic amides include polyethylene polyvinyl alcohol copolymers; and polyvinyl caprolactam-polyvinyl acetate-polyethylene glycol graft copolymer (SOLUPLUS®, BASF Corp.). Commercially available copolymers of vinyl pyrrolidone and vinyl acetate include PLASDONE® S630 (Ashland, Inc., Covonton, Ky.) and KOLLIDON® VA 64 (BASF Corp., Florham Park, N.J.), which contain vinyl pyrrolidone and vinyl acetate in a 60:40 ratio. Other copolymers of vinyl pyrrolidone and vinyl acetate can also be used in the invention. In one embodiment, the copolymer contains at least 40% vinyl pyrrolidone. Smaller amounts of vinyl pyrrolidone can also be utilized.

Absorption enhancing polymers also include cellulosic polymers. Cellulosic polymers include cellulose esters or cellulose ethers, such as alkylcelluloses (e.g., methylcellulose or ethylcellulose), hydroxyalkylcelluloses (e.g., hydroxypropylcellulose), hydroxyalkylalkylcelluloses (e.g., hydroxypropylmethylcellulose), and cellulose phthalates or succinates (e.g., cellulose acetate phthalate and hydroxypropylmethylcellulose phthalate, hydroxypropylmethylcellulose succinate, or hydroxypropylmethylcellulose acetate succinate); cellulose esters or cellulose ethers, such as alkylcelluloses (e.g., methylcellulose or ethylcellulose), hydroxyalkylcelluloses (e.g., hydroxypropylcellulose), hydroxyalkylalkylcelluloses (e.g., hydroxypropylmethylcellulose), and cellulose phthalates or succinates (e.g., cellulose acetate phthalate and hydroxypropylmethylcellulose phthalate, hydroxypropylmethylcellulose succinate, or hydroxypropylmethylcellulose acetate succinate (HPMCAS)). Commercially available examples of these include hydroxypropyl methylcellulose (HPMC) E3, HPMC E5, HPMC E6, HPMC E15, HPMC K3, HPMC A4, HPMC A15, HPMC acetate succinate (AS) LF, HPMC AS MF, HPMC AS HF, HPMC AS LG, HPMC AS MG, HPMC AS HG, HPMC phthalate (P) 50, and HPMC P 55.

When specific polymers that are suitable for use in the compositions of the present invention are blended, the blends of such polymers may also be suitable. Thus the term “polymer” is intended to include blends of polymers in addition to a single species of polymer.

The action of absorption enhancing polymers may be improved by the presence of a surfactant. Thus, compositions of the present invention may optionally comprise one or more surfactants. The surfactants may increase the rate of dissolution by facilitating wetting, thereby increasing the maximum concentration of dissolved drug. The surfactants may also make the dispersion easier to process. Surfactants may also stabilize the amorphous dispersions by inhibiting crystallization or precipitation of the drug by interacting with the dissolved drug by such mechanisms as complexation, formation of inclusion complexes, formation of micelles, and adsorption to the surface of the solid drug. Surfactants may also facilitate absorption of APIs by altering API permeability and/or efflux directly. See, e.g., Yu et al., Pharm Res, 1999, 16:1812-7. Non-limiting examples of pharmaceutically acceptable surfactants that may be suitable for the present invention include polyoxyethylene castor oil derivates, e.g. polyoxyethyleneglycerol triricinoleate or polyoxyl 35 castor oil (CREMOPHOR® EL; BASF Corp.) or polyoxyethyleneglycerol oxystearate such as polyethylenglycol 40 hydrogenated castor oil (CREMOPHOR® RH 40, also known as polyoxyl 40 hydrogenated castor oil or macrogolglycerol hydroxystearate) or polyethylenglycol 60 hydrogenated castor oil (CREMOPHOR® RH 60); or a mono fatty acid ester of polyoxyethylene sorbitan, such as a mono fatty acid ester of polyoxyethylene (20) sorbitan, e.g. polyoxyethylene (20) sorbitan monooleate (Tween 80), polyoxyethylene (20) sorbitan monostearate (Tween 60), polyoxyethylene (20) sorbitan monopalmitate (Tween 40), or polyoxyethylene (20) sorbitan monolaurate (Tween 20). Other non-limiting examples of suitable surfactants include polyoxyethylene alkyl ethers, e.g. polyoxyethylene (3) lauryl ether, polyoxyethylene (5) cetyl ether, polyoxyethylene (2) stearyl ether, polyoxyethylene (5) stearyl ether; polyoxyethylene alkylaryl ethers, e.g. polyoxyethylene (2) nonylphenyl ether, polyoxyethylene (3) nonylphenyl ether, polyoxyethylene (4) nonylphenyl ether, polyoxyethylene (3) octylphenyl ether; polyethylene glycol fatty acid esters, e.g. PEG-200 monolaurate, PEG-200 dilaurate, PEG-300 dilaurate, PEG-400 dilaurate, PEG-300 distearate, PEG-300 dioleate; alkylene glycol fatty acid mono esters, e.g. propylene glycol monolaurate (lauroglycol, such as lauroglycol FCC); sucrose fatty acid esters, e.g. sucrose monostearate, sucrose distearate, sucrose monolaurate, sucrose dilaurate; sorbitan fatty acid mono esters such as sorbitan mono laurate (Span 20), sorbitan monooleate, sorbitan monopalnitate (Span 40), or sorbitan stearate; D-alpha-tocopheryl polyethylene glycol 1000 succinate (vitamin E TPGS); or a combination or mixture thereof. Other suitable surfactants may include, but are not limited to, block copolymers of ethylene oxide and propylene oxide, also known as polyoxyethylene polyoxypropylene block copolymers or polyoxyethylene polypropyleneglycol, such as POLOXAMER® 124, POLOXAMER® 188, POLOXAMER® 237, POLOXAMER® 388, or POLOXAMER® 407 (BASF Corp.). As described above, a mixture of surfactants may be used in a solid composition of the present invention. In one embodiment, the surfactant is selected from sodium lauryl sulfate (SLS) and vitamin E TPGS.

The relative amount of drug, polymer and surfactant can vary widely. The optimal amount of the polymer and surfactant can depend, for example, on the hydrophilic lipophilic balance (HLB), melting point, and water solubility of the copolymer, and the surface tension of aqueous solutions of the surfactant.

The compositions of the invention comprise an effective amount of Compound 1, or a pharmaceutically acceptable salt thereof, but comprise less than about 50% w/w of Compound 1 due to the poor absorption seen with formulations having greater than about 50% w/w of Compound 1. Thus, in some embodiments, the concentration of Compound 1 can vary from about 0.1% to about 40.0%, from about 5.0% to about 35.0%, or from about 10% to about 30%, by weight based on the total combined weight of the drug substance, water-soluble polymer, and optional surfactant (not including other excipients).

The concentration of the surfactant, if present, can vary from about 2.0% to about 15% or about 5.0% to about 10% by weight based on the total combined weight of the drug substance, water-soluble polymer, and surfactant (not including other excipients).

The concentration of the absorption enhancing polymer is added to the concentrations of the Compound 1 and optional surfactant to add up to 100%. The concentration can vary from about 0.01% to about 90%, from about 1% to about 75%, from about 10% to about 60%, and from about 10% to about 55% by weight based on the total combined weight of the drug substance and polymer, not including other excipients.

An embodiment of the present invention is directed to a composition that comprises from between about 0.1% to about 40% of Compound 1, or a pharmaceutically acceptable salt thereof, about 2.0% to about 15% or about 5% to about 10% surfactant, with the balance of the formulation being the water-soluble polymer.

The compositions described above relate to compositions produced by solvent removal (e.g., spray-drying), introduction of an antisolvent (e.g., precipitation), addition of heat together with mixing (e.g., extrusion), mechanical activation or other means (e.g., to produce a “solid dispersion intermediate”).

The solid dispersions of the present invention are prepared by processes that are suitable for causing Compound 1 to form a dispersion (also referred to as an amorphous dispersion) in the polymer such that the compound is generally amorphous or dissolved in the polymer or a component of the composition, such as a surfactant. The dispersions are stable, and the compound does not form crystals or other insoluble particles. Such methods include solution methods, such as spray drying, spray coating, freeze-drying, and evaporation of a co-solvent under vacuum or by heating a solution of polymer and compound. Such methods also include methods that blend the solid compound with the polymer in the molten state, such as hot melt extrusion, and methods of compounding the solid non-molten polymer and compound under heat and pressure to form a dispersion. If the dispersion is effectively a homogeneous molecular dispersion of the individual components, it may also be described as a solid solution, a specific subclass of solid dispersions.

Processes for making solid dispersions of Compound 1 with a water-soluble polymer and optional surfactant include (a) extrusion, e.g., hot melt extrusion; and (b) spray drying from a solution or suspension. Both of these processes are well known in the art.

Spray drying is well known (see, e.g., Masters, Spray Drying Handbook, 1991, 5^{th} edition, Longman Scientific & Technical) and widely practiced in a variety of industrial applications, including spray drying of milk (see, e.g., U.S. Pat. No. 4,187,617) and pharmaceutical products (see, e.g., U.S. Pat. No. 6,763,607). In spray drying, the polymer, Compound, and, possibly also, a surfactant, are dissolved in a solvent and are then sprayed through a nozzle as a fine spray into a chamber where the solvent is evaporated quickly to make particles comprising polymer, drug, and optional surfactant. Ideally, the solvent is any solvent in which all of the components of the composition are soluble and which is readily evaporated in a spray dryer. The solvent should also be suitable for use in preparing pharmaceutical compositions. In certain embodiments of the invention, the use of mixed solvent systems, particularly those containing a combination of water and a volatile solvent, are necessary to facilitate the production of solid solution intermediates containing Compound 1, an absorption enhancing polymer or polymer(s), and, optionally, a surfactant. Useful volatile solvents for spray drying include acetone, ethanol, methanol, dichloromethane, isopropanol and tetrahydrofuran (THF). In an embodiment of the present invention, spray drying occurs in a mixed solvent system comprising a volatile solvent and a non-volatile solvent. In one embodiment, the volatile solvent is ethanol, methanol or acetone. In one embodiment, the non-volatile solvent is water. An exemplary mixed solvent system is acetone:water (90:10). Mixed solvent systems are described in International Patent Application Publication No. WO2007/109605 and U.S. Patent Application Publication No. US2007/0026083.

Following formation of a solid dispersion, the resulting spray dried intermediate can undergo a secondary drying step to remove residual solvents. This secondary drying unit operation can occur in a static dryer or an agitated dryer. Gas, humidified gas, or vacuum may be applied to the material in the secondary dryer and such application can be useful in more rapidly removing residual solvents that remain in the spray dried intermediate. See, e.g., European Patent Application No. EP1855652 A2 (and references therein) and International Patent Application Publication No. WO2008012617A1 (and references therein).

In hot melt extrusion, the polymer, drug, and optional surfactant may be either premixed together (e.g., via a wet granulation process) or fed as independent feed streams into the extruder (see Polymer Extrusion 4^{th} Edition by Chris Rauwendaal 2001, Hanser Gardner Publications, Inc., Cincinnati, Ohio or Schenck et al., (2010), Achieving a Hot Melt Extrusion Design Space for the Production of Solid Solutions, in Chemical Engineering in the Pharmaceutical Industry: R&D to Manufacturing (ed. D. J. am Ende), John Wiley & Sons, Inc., Hoboken, N.J., USA). In accordance with this embodiment, any means for preparing a melt in any convenient apparatus in which an admixture of Compound 1, an absorption enhancing polymer and, optionally a surfactant can be heated and optionally mixed can be used. Solidification can be carried out by merely cooling the melt. Once a solid is obtained, the solid can be further mechanically processed to provide a convenient form for incorporation into a medicament, for example, tablets or capsules.

It will be appreciated that other methods of preparing a melt, solidifying it, and forming the solid into conveniently sized particles can be utilized without departing from the spirit of the invention. For example, compositions of the invention may be prepared using an extruder. When an extruder is employed to prepare compositions of the invention, the material may be introduced into the extruder either in a pre-flux state, that is, as a dry admixture, or in a fluxed state, that is in a melted, plastic, or semi-solid state achieved after the application of sufficient heat to the admixture to cause Compound 1 to dissolve in the polymer. Optionally when a fluxed charge is prepared, blending may be employed during heating to promote uniformity of the fluxed material.

If the material is introduced to the extruder in a fluxed state, residence time in the extruder is selected to be just sufficient to ensure homogeneity of the composition, and the temperature is preferably maintained in the extruder at a level just sufficient to insure that the material maintains its plasticity so that it can be extruded into a conveniently shaped extrudate. If the material is introduced into an extruder in a pre-flux state, the extruder components, for example, the barrels and any mixing chamber present in the equipment, will be maintained at a temperature sufficient to promote fluxing of the admixture. Temperatures selected for use in processing a composition will also take into account that blending which occurs within the extruder equipment, for example, in a mixing section of the barrels, will also contribute to localized fluxing of the admixture by imparting shear-stresses that induce heating in the mixture. Additionally it will be appreciated that equipment temperatures and residence times will be selected to minimize the amount of time that the admixture placed into the extruder spends under conditions of heating and/or shear stress so as to minimize the amount of Compound 1 which is decomposed during formation of the composition, as discussed above. In general, extrusion processes in which heating is applied to the material extruded are termed “hot-melt/extrusion processes.” When compositions of the present invention are prepared using extrusion equipment, the extrudate thus provided can be in any convenient shape, for example, noodles, cylinders, bars, or the like. If desired, the extrudate can be further processed, for example by milling, to provide a particulate form of the composition.

Pharmaceutical compositions intended for oral use may be prepared from the solid dispersions described above in accordance with the methods described herein and other methods known to the art for the manufacture of pharmaceutical compositions. Such compositions may further contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be for example, inert diluents, such as mannitol, microcrystalline cellulose, calcium carbonate, sodium carbonate, lactose, calcium phosphate or sodium phosphate; granulating and disintegrating agents, for example, croscarmellose sodium, sodium chloride, corn starch, or alginic acid; binding agents, for example starch, gelatin or acacia; glidants such as colloidal silicon dioxide, lubricating agents, for example magnesium stearate, sodium stearyl fumarate, stearic acid or talc, and antioxidants, for example, propyl gallate, butylated hydroxyanisole and nutylated hydroxy toluene. The tablets may be uncoated or they may be coated to delay disintegration and absorption in the gastrointestinal tract and thereby provide a sustained action over a longer period. Compositions for oral use may also be presented as capsules (e.g., hard gelatin) wherein the active ingredient is mixed with an inert solid diluent, for example, calcium carbonate, calcium phosphate or kaolin, or as soft gelatin capsules wherein the active ingredient is mixed with liquids or semisolids, for example, peanut oil, liquid paraffin, fractionated glycerides, surfactants or olive oil. Aqueous suspensions contain the active materials in admixture with excipients suitable for the manufacture of aqueous suspensions. Dispersible powders and granules suitable for preparation of an aqueous suspension by the addition of water provide the active ingredient in admixture with a dispersing or wetting agent, suspending agent and one or more preservatives. In certain embodiments of the invention, the pharmaceutical compositions of the invention include a diluent system, lubricant and glidant, at concentrations of about 0-70% w/w, about 0.25-3.0% w/w, about 0-3.0% w/w, respectively, or at about 20-60% w/w, about 0.5-2.0%, and about 0-1.0% w/w, respectively. In certain embodiments of the invention, the solid dispersion is blended with a diluent, one or more disintegrating agents, lubricant and glidant. An exemplary formulation includes mannitol, croscarmellose sodium, sodium chloride, colloidal silica and magnesium stearate.

The disintegrant (e.g., croscarmellose sodium, sodium starch glycolate, etc.) is present in a concentration from about 5-20% or about 5-10%. A salt is also present, which may be sodium chloride, potassium chloride or a combination thereof. The combination of salt and disintegrant is present at a concentration from about 5% to about 35% w/w of the final pharmaceutical composition. Applicants have surprisingly found that pharmaceutical compositions comprising these levels of disintegrant and salt (in combination with absorption enhancing polymer) provides a rapidly disintegrating dosage form. Rapidly disintegrating tablets based on solid dispersions are disclosed in U.S. Pat. No. 7,189,415.

The blended material may be roller compacted or wet granulated to densify and/or reduce the risk of segregation of components during subsequent handling (e.g., compression into tablets). Granulation steps can also be used to minimize the impact of raw material property variability (e.g., excipient particle size) on subsequent processing (e.g., tablet compression) and ultimate product performance. Lubrication is typically performed prior to roller compaction and tablet compression to reduce the tendency of material to adhere to compression surfaces (e.g., tablet tooling). A preferred lubricant is magnesium stearate. These methods can be carried out by those skilled in the art. See, e.g., Ansel, Introduction to Pharmaceutical Dosage Forms, Seventh Edition, 1999.

To prepare the pharmaceutical compositions of the invention, the solid dispersion is compressed into an oral dosage form including tablets or capsules. Tablets can be prepared with a variety of possible shapes (ellipsoidal, capsule, biconvex round, etc.). The powder can also be encapsulated in capsule dosage (e.g., using hard gelatin capsules). Techniques suitable for preparing solid oral dosage forms of the present invention are described in Remington's Pharmaceutical Sciences, 18th edition, edited by A. R. Gennaro, 1990, Chapter 89 and in Remington—The Science and Practice of Pharmacy, 21st edition, 2005, Chapter 45. In certain embodiments, the solid dispersion is present in an amount of about 5-70% w/w of the pharmaceutical composition or about 20-50% w/w of the final pharmaceutical composition.

For combination regimens, other drug substance(s) can be added to the solid solution or the tablet formulation, either in a crystalline form, neat amorphous form, or as a solid solution. Exemplary drug substances include, but are not limited to, HCV protease inhibitors, HCV polymerase inhibitors, HCV NS4A inhibitors and HCV NS5A inhibitors.

HCV protease inhibitors include, but are not limited to, those disclosed in U.S. Pat. Nos. 7,494,988, 7,485,625, 7,449,447, 7,442,695, 7,425,576, 7,342,041, 7,253,160, 7,244,721, 7,205,330, 7,192,957, 7,186,747, 7,173,057, 7,169,760, 7,012,066, 6,914,122, 6,911,428, 6,894,072, 6,846,802, 6,838,475, 6,800,434, 6,767,991, 5,017,380, 4,933,443, 4,812,561 and 4,634,697; U.S. Patent Application Publication Nos. US20020068702, US20020160962, US20050119168, US20050176648, US20050209164, US20050249702 and US20070042968; and International Patent Application Publication Nos. WO 03/006490, WO 03/087092, WO 04/092161 and WO 08/124148.

HCV protease inhibitors also include, but are not limited to, SCH503034 (Boceprevir, Schering-Plough), SCH900518 (Schering-Plough), VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott), TMC-435350 (Medivir), ITMN-191/R7227 (InterMune/Roche), EA-058 (Abbott/Enanta), EA-063 (Abbott/Enanta), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), BMS-650032 (Bristol-Myers Squibb), VX-985 (Vertex) and PHX1766 (Phenomix).

Further examples of HCV protease inhibitors include, but are not limited to, those disclosed in Landro et al., Biochemistry, 36(31):9340-9348 (1997); Ingallinella et al., Biochemistry, 37(25):8906-8914 (1998); Llinàs-Brunet et al., Bioorg Med Chem Lett, 8(13):1713-1718 (1998); Martin et al., Biochemistry, 37(33):11459-11468 (1998); Dimasi et al., J Virol, 71(10):7461-7469 (1997); Martin et al., Protein Eng, 10(5):607-614 (1997); Elzouki et al., J Hepat, 27(1):42-48 (1997); BioWorld Today, 9(217):4 (Nov. 10, 1998); U.S. Patent Application Publication Nos. US2005/0249702 and US 2007/0274951; and International Patent Application Publication Nos. WO 98/14181, WO 98/17679, WO 98/17679, WO 98/22496, WO 99/07734 and WO 05/087731.

HCV polymerase inhibitors include, but are not limited to, VP-19744 (Wyeth/ViroPharma), PSI-7851 (Pharmasset), GS-7977 (sofosbuvir, Gilead), R7128 (Roche/Pharmasset), PF-868554/filibuvir (Pfizer), VCH-759 (ViroChem Pharma), HCV-796 (Wyeth/ViroPharma), IDX-184 (Idenix), IDX-375 (Idenix), NM-283 (Idenix/Novartis), R-1626 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3281 (Merck), VCH222 (ViroChem), VCH916 (ViroChem), VCH716(ViroChem), GSK-71185 (Glaxo SmithKline), ANA598 (Anadys), GSK-625433 (Glaxo SmithKline), XTL-2125 (XTL Biopharmaceuticals), and those disclosed in International Patent Application Publication Nos. WO 13/177219, WO 14/058801, WO 14/058801, WO 14/062596, and WO 12/142085, US Patent Application Publication Nos. 20140206640 and 20140161770, Ni et al., Current Opinion in Drug Discovery and Development, 2004, 7(4):446; Tan et al., Nature Reviews, 2002, 1:867; and Beaulieu et al., Current Opinion in Investigational Drugs, 2004, 5:838.

HCV NS4A inhibitors include, but are not limited to, those disclosed in U.S. Pat. Nos. 7,476,686 and 7,273,885; U.S. Patent Application Publication No. US20090022688; and International Patent Application Publication Nos. WO 2006/019831 and WO 2006/019832. Additional HCV NS4A inhibitors include, but are not limited to, AZD2836 (Astra Zeneca) and ACH-806 (Achillon Pharmaceuticals, New Haven, Conn.).

HCV NS5A inhibitors include, but are not limited to, those disclosed in U.S. Pat. Nos. 8,871,759 and 8,609,635, and International Patent Application Publication Nos. WO 14/110705 and WO 14/110706.

In one embodiment, the drug substance is a compound disclosed in U.S. Patent Application Publication No. US20120083483. In one aspect of this embodiment, the drug substance is

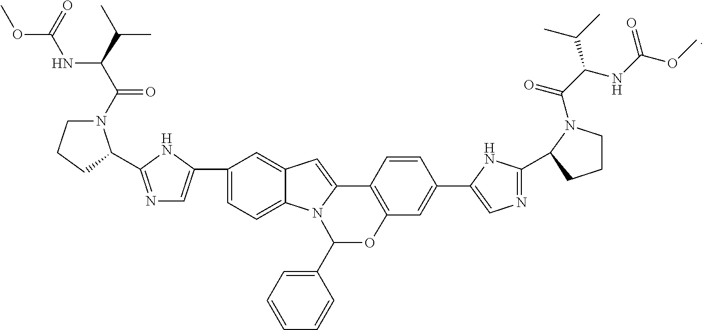

Actual dosage levels of active ingredients in the pharmaceutical compositions of the invention may be varied to obtain an amount of active ingredient that is effective to obtain a desired therapeutic response for a particular composition and method of administration. The selected dosage level therefore depends upon the desired therapeutic effect, the route of administration, the potency of the administered drug, the desired duration of treatment, and other factors. Compound 1 can be administered orally in a dosage range of about 0.001 to about 1000 mg/kg of mammal (e.g., human) body weight per day in a single dose or in divided doses. One dosage range is about 0.01 to about 500 mg/kg body weight per day orally in a single dose or in divided doses. Another dosage range is about 0.1 to about 100 mg/kg body weight per day orally in single or divided doses. For oral administration, the compositions can be provided in the form of tablets or capsules containing about 1.0 mg to about 500 mg or about 25 mg to about 100 mg of the active ingredient, particularly about 1, 5, 10, 15, 20, 25, 35, 40, 50, 55, 60, 75, 80, 100, 150, 200, 250, 300, 400, 500, and 750 mg of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The following examples serve only to illustrate the invention and its practice. The examples are not to be construed as limitations on the scope or spirit of the invention.

### EXAMPLES

Example 1

### Conventional Wet Granulation of Vitamin E TPGS

The pharmacokinetics of a conventional wet granulation of Compound 1 with Vitamin E TPGS was examined. As described below, a crystalline potassium salt form of Compound 1, as described in International Patent Application Publication No. WO2013/028465, was wet granulated, dried, and filled into capsules to produce the formulation with a composition outlined in the Table 1 below. A blend of all the dry components in Table 1 was prepared using the Turbula mixer at 46 RPM for 100 revolutions. An aqueous solution (16% w/w) of Vitamin E TPGS was prepared and added drop by drop with a syringe to a blended bed of the blend. The components were mixed using a mortar and pestle until visibly granulated. The wet granulation was dried overnight at 40° C. The extra-granular magnesium stearate was blended together with the dried granulation using the Turbula mixer at 46 RPM for 50 revolutions to make a formulation that was hand-filled into capsules by weight.

TABLE 1
Composition of Formulation 1

| | Amount |
|---|---|
| Components | (% w/w) |
| Compound 1 (K-salt) | 21.00 |
| AVICEL ® PH-101 | 20.00 |
| Potassium Carbonate | 40.00 |
| Lactose Monohydrate 312 | 7.00 |
| Vitamin E TPGS | 5.00 |
| Hydroxypropyl cellulose EXF | 3.00 |
| Croscarmellose, Na | 3.00 |
| Magnesium Stearate (intra-granular) | 0.50 |
| Magnesium Stearate (extra-granular) | 0.50 |

These capsules were administered to male beagle dogs pretreated with pentagastrin to minimize the pH variability of the dog's stomach (pentagastrin was administered intramuscularly at a target dose level of 6 μg/kg, 0.05 mL/kg, 30±5 minutes prior to dosing). The exposure of this formulation of Compound 1 was compared to a reference formulation of Compound 1 completely dissolved in a solution. The PEG 400 solution reference formulation was prepared by dissolving the potassium salt form of Compound 1 in PEG 400 to a solution concentration of 6 mg/mL (free acid basis) and dosing this solution at 2 mL/kg in dogs. The male beagle dogs (weighing 11-14 kg) were selected and fasted overnight prior to dosing. Water was removed before dosing and was returned 1 hour after dosing. The dogs were dosed with Formulation 1 and received a 3.5 mL/kg water rinse via oral gavage following dosing. Food was returned at 4 hours after dosing. A 1 mL blood sample was drawn from the jugular vein into EDTA (ethylenediaminetetraacetic acid) tubes at pre-dose and 0.25, 0.5, 1, 2, 4, 6, 8, and 24 hours after dosing. The plasma was separated by centrifugation (10 minutes at 2800 rpm) and kept frozen at −70° C. until analysis. Concentrations of Compound 1 in dog plasma were quantified by LC-MS/MS (liquid chromatography tandem mass spectrometry) analysis. Area under the curve (AUC₀₋ₐₗₗ), observed maximum plasma concentration (Cₘₐₓ), time of Cₘₐₓ (Tₘₐₓ), means and SE were calculated using a linear trapezoidal, non-compartmental model of WinNonLin v5.01. Plasma profiles were generated using EXCEL® 2002 SP3 and WinNonLin v5.01. The area under the plasma concentration curve (AUC) from Formulation 1 is less than 5% of that of the reference formulation indicating poor absorption. The results are shown in Table 2.

TABLE 2
Pharmacokinetic data for Compound 1 following oral administration
to male beagle dogs (fasted state; 100 mg; pentagastrin was
administered intramuscularly at a target dose level of 6 μg/kg,
0.05 mL/kg 30 ± 5 minutes prior to dosing)

| | AUC₀₋₂₄ hr | | |
|---|---|---|---|
| Formulation | (μM * hr) | Cₘₐₓ (μM) | Tₘₐₓ (hr) |
| PEG 400 solution | 294 ± 32.2 | 30.1 ± 2.55 | 3.0 (2.0-4.0) |
| (6 mg/mL) (n = 6) | | | |
| Formulation 1 (n = 5) | 1.60 ± 0.714 | 0.379 ± 0.169 | 2.0 (2.0-4.0) |

This example illustrates the poor absorption obtained using a crystalline salt form of Compound 1 instead of a solid solution based formulation.

### Example 2

### Neat Amorphous Based Formulations

30.8% w/w Compound 1, 0.1% w/w butylated hydroxyanisole, and 0.1% w/w butylated hydroxy toluene was spray dried from an acetone solution to produce a spray dried amorphous form of Compound 1. A Niro PSD-1 spray dryer with a pressure nozzle was used to produce the spray dried particles. Heated nitrogen was supplied to the spray dryer at an inlet temperature sufficient to maintain a 43° C. outlet temperature and a gas flow rate of 1750 g/min. The spray drying solution flow rate was 200-300 g/min which required a nozzle pressure of approximately 190 PSI. This amorphous spray dried intermediate of Compound 1 was formulated into tablets and capsule formulations with the compositions outlined in Tables 3 and 4 below.

TABLE 3
Composition of Formulation 2

| | Amount |
|---|---|
| Components | (mg/tablet) |
| Compound 1 (free acid) | 200.0 |
| Butylated Hydroxyanisole | 0.5001 |
| Butylated Hydroxy Toluene | 0.5001 |
| Sodium Lauryl Sulfate | 14.29 |
| Microcrystalline Cellulose | 29.49 |
| Lactose Monohydrate | 29.49 |
| Croscarmellose Sodium | 8.570 |
| Magnesium Stearate (non-bovine) | 2.860 |
| Total Tablet Weight | 285.7 |

TABLE 4
Composition of Formulation 3

| Components | mg/capsule |
|---|---|
| Compound 1 (free acid) | 200.0 |
| Butylated Hydroxyanisole | 0.5001 |
| Butylated Hydroxy Toluene | 0.5001 |
| Sodium Lauryl Sulfate | 11.00 |
| Capsule, Gelatin, Size 1, White Opaque | 76 |
| Total Capsule Weight | 288.0 |

Formulation 2 was prepared by blending of the spray dried intermediate (italicized in Table 3) with the components in Table 3 except magnesium stearate using a 5 L Twin Shell Blender for 10 minutes at 30 RPM. Half of the magnesium stearate (screened through No. 60 mesh) was then added to the blender and the mixture was lubricated for 5 minutes at 30 RPM. The blend was then granulated into ribbons using an Alexanderwerk WP 120 Roller Compactor with a 40 mm knurled roll operating at a roll pressure of 2.5 MPa with a roll gap of 2.0 mm. The ribbons were subsequently milled using the rotary fine granulator equipped with 2.0 mm and 1.0 mm size CONIDUR® screens. The granules were then lubricated with the remaining magnesium stearate (screened through No. 60 mesh) in the Twin Shell Blender for 5 minutes at 30 RPM. The lubricated granules were then compressed on a rotary tablet press to a 285.7 mg image tablet using 2×11/32″ round standard concave tooling. The hardness of the tablets was measured to be between 4.5 to 11.7 kiloponds (kp=1 kgf).

Formulation 3 was prepared by blending the spray dried intermediate (italicized in Table 4) with sodium lauryl sulfate using a 1 L Twin Shell Blender for 20 minutes at 30 RPM. The blend was filled into capsules using a semi-automatic capsule filling machine with a 150 unit plate at a target fill weight of 212.0 mg into a size 1 HG capsule.

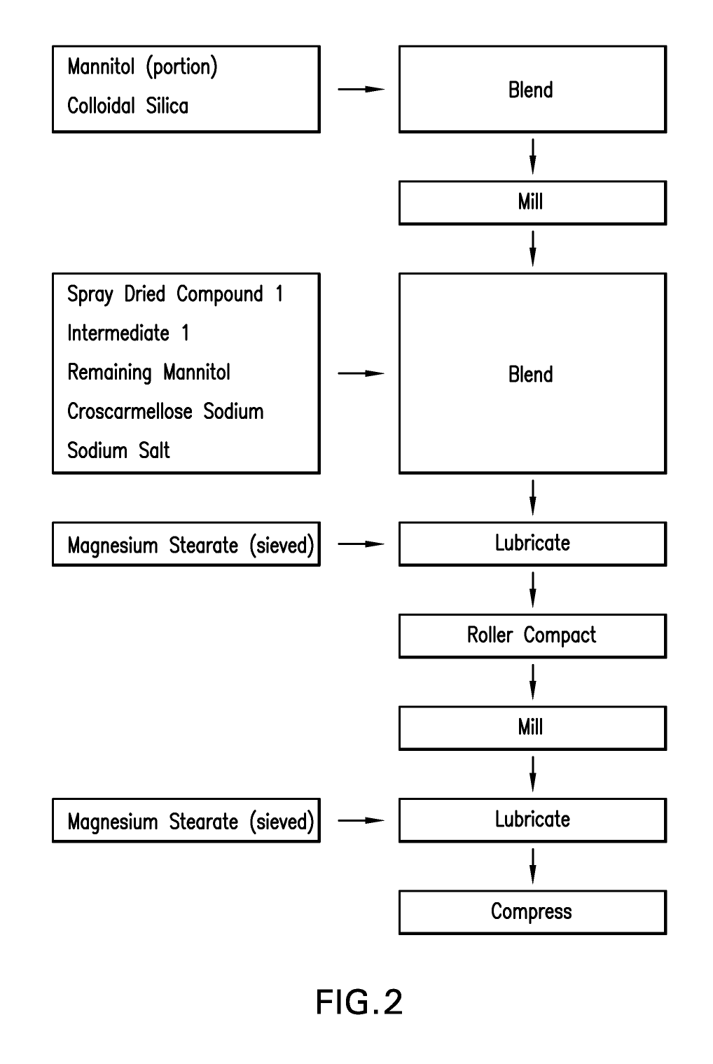

Formulation 4 is a tablet composition (Table 5) based on a spray dried intermediate of Compound 1 dispersed into copovidone and sodium lauryl sulfate (SLS). FIG. 2 illustrates the process used to produce Formulation 4. To produce Formulation 4, Compound 1, copovidone, and SLS were dissolved into a 90/10 (w/w) acetone/water solution. This spray drying solution was prepared such that it contains 20% w/w solids in solution. The spray drying solution was then pumped through a spray drying nozzle (e.g., a pressure nozzle) in order to produce a plume of atomized particles. These particles were dried in a chamber that can contain an inert heated gas (e.g., nitrogen). The particles thus produced were collected (e.g., using a cyclone). Typically, a secondary drying operation was used to sufficiently dry the spray dried intermediate. Humid nitrogen or air may be used to facilitate drying. Tray dryers or agitated dryers can be used to perform this secondary drying operation. The dried spray dried intermediate (option to screen through No. 30 mesh) was added to the “downstream tablet” components listed in Table 5, except the magnesium stearate, where the colloidal silica and a portion of mannitol were co-screened with a Quadro Comill equipped with a round impeller and 32 R screen, processed at 2000 RPM, and the remaining components may be screened through a No. 30 mesh and blended using a 600 L Bohle Blender for 21 minutes at 6 RPM. One-third of the magnesium stearate (screen through No. 60 mesh) was added to the blender and the mixture was lubricated for 6 minutes at 10 RPM. The blend was then granulated into ribbons using an Alexanderwerk WP 120 Roller Compactor with a 40 mm knurled roll operating at a roll pressure of 29-39 bar with a roll gap of 2.0 mm. The ribbons were subsequently milled using the rotary fine granulator equipped with 2.0 mm and 1.0 mm size CONIDUR® screens. The granules were then lubricated with the remaining magnesium stearate (screened through No. 60 mesh) in the 60 L Bohle blender for 6 minutes at 10 RPM. The lubricated granules were then compressed on a rotary tablet press to a 1000 mg image tablet using 16-24 tablet stations with size 7.94 mm×19.05 mm caplet tooling. The hardness of the tablets was measured to be between 15 to 25 kiloponds (kp=1 kgf).

TABLE 5
Composition of Formulation 4

| | |
|---|---|
| | Amount |
| Components | (mg/tablet) |

Spray Dried Intermediate

| | |
|---|---|
| Compound 1 (free acid) | 100.0 |
| Polyvinylpyrolidone/Vinyl Acetate Copolymer | 214.2 |
| (Kollidon VA64) | |
| Butylated Hydroxyanisole | 0.8333 |
| Butylated Hydroxy Toluene | 0.8333 |
| Propyl Gallate | 0.8333 |
| Sodium Lauryl Sulfate | 16.67 |

Downstream Tablet

| | |
|---|---|
| Mannitol | 449.2 |
| Croscarmellose Sodium | 100.0 |
| Sodium Chloride | 100.0 |
| Colloidal Silicon Dioxide | 2.500 |
| Magnesium Stearate (non-bovine) | 15.00 |
| Total | 1000 |

The oral absorption obtained from Formulations 2 and 3 was evaluated as part of a human clinical study. In this study, Formulations 2 and 3 were compared to a reference formulation, Formulation 4. The results are shown in Table 6.

TABLE 6
Summary of Human PK Results from Biocomparison Study
(600 mg dose; healthy subjects)

| Comparison | AUC_{0-∞}^{\|\|} | Cₘₐₓ^{\|\|} | C₂₄^{\|\|} |
|---|---|---|---|
| Formulation 2/ | 0.15 (0.12, 0.19) | 0.05 (0.03, 0.08) | 0.53 (0.47, 0.60) |
| Formulation 4 | | | |
| Formulation 3/ | 0.23 (0.19, 0.29) | 0.11 (0.07, 0.16) | 0.59 (0.52, 0.66) |
| Formulation 4 | | | |

^{||}GMR (90% CI).
GMR = Geometric least-squares mean ratio between formulations;
CI = Confidence interval.

The exposure of Compound 1 following the oral administration of both Formulation 2 and Formulation 3 is significantly less than that of Formulation 4 (Table 6). This example illustrates that not all amorphous presentations of Compound 1 provide sufficient absorption. In particular, Formulations 2 and 3, in which Compound 1 is formulated as an undispersed (e.g., neat) component, provide less than ⅓ of the exposure when orally administered as compared with Formulation 4.

### Example 3

### Copovidone-SLS Formulations

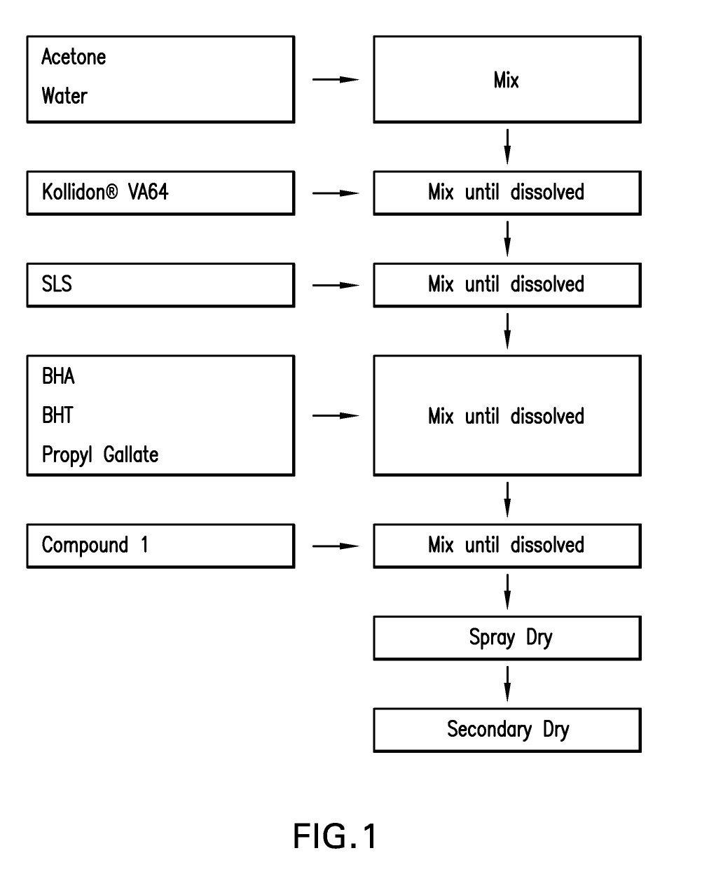

Formulations were prepared from solid solutions of Compound 1, SLS, and copovidone by spray drying from a 90/10 (w/w) acetone/water solvent system in a process as described by FIG. 1. The three solid components of the spray drying solution were incorporated into the solution at 20% w/w. A Niro PSD-2 spray dryer with a pressure nozzle was used to produce the spray dried particles. Heated nitrogen was supplied to the spray dryer at an inlet temperature sufficient to maintain a 52° C. outlet temperature and a gas flow rate of 7500 g/min. The spray drying solution flow rate was 700-800 g/min which required a nozzle pressure of approximately 400 PSI.

A tablet composition (Formulation 5) was prepared with a composition identical to that described in Table 5 and using a similar process as illustrated in FIG. 2, but resulting in a tablet of half the size (500 mg vs. 1000 mg). Additional tablet compositions (Formulations 6-9) were prepared using different levels and types of salt (NaCl, KCl, CaCl₂, etc.), grades of salt (coarse vs. fine, mean particle size of approximately 380 μm and 185 μm, respectively), and disintegrant (croscarmellose sodium, sodium starch glycolate, etc.). A master blend of the spray dried intermediate, mannitol and colloidal silica was prepared by co-sieving materials through a No. 30 mesh and blending using a Turbula blender for 5 minutes at 46 RPM. Appropriate amounts of the relevant disintegrant and/or salt were sieved through a No. 30 mesh and blended with a portion of the master blend using a Turbula blender for 5 minutes at 46 RPM. Magnesium stearate (sieved through No. 60 mesh) was added to the blends and the mixture was lubricated using the Turbula blender for 2 minutes at 46 RPM. The tablet compositions were made by compressing the powder blend containing the spray dried solid dispersion into tablets using a 16/32″ round standard concave tooling on small scale single station compression equipment (Carver, MTS or Lloyds). The tablet hardness measurements are listed in Table 7. The disintegration time of the resulting tablet compositions was measured using a standard USP reciprocating disintegration apparatus with cylinders in 900 mL of Simulated Gastric Fluid (1 L water, 1.4 mL concentrated 0.1N HCl, 2.0 g NaCl) at 37° C. Table 7 summarizes the measured disintegration behavior of these formulations compared with the reference (Formulation 4).

TABLE 7
Composition of Formulations 5-9

| | | | | | |
|---|---|---|---|---|---|
| | Formulation | Formulation 6 | Formulation | Formulation | Formulation |
| Components | 5 (mg/tab) | (mg/tab) | 7 (mg/tab) | 8 (mg/tab) | 9 (mg/tab) |

Spray Dried Intermediate

| | | | | | |
|---|---|---|---|---|---|
| Compound 1 (free | 50 | 50 | 50 | 50 | 50 |
| acid) | | | | | |
| Polyvinylpyrolidone/ | 107.1 | 107.1 | 107.1 | 107.1 | 107.1 |
| Vinyl Acetate | | | | | |
| Copolymer | | | | | |
| (KOLLIDON ® VA64) | | | | | |
| Butylated | 0.4167 | 00.4167 | 0.4167 | 0.4167 | 0.4167 |
| Hydroxyanisole | | | | | |
| Butylated Hydroxy | 0.4167 | 0.4167 | 0.4167 | 0.4167 | 0.4167 |
| Toluene | | | | | |
| Propyl Gallate | 0.4167 | 0.4167 | 0.4167 | 0.4167 | 0.4167 |
| Sodium Lauryl | 8.335 | 8.335 | 8.335 | 8.335 | 8.335 |
| Sulfate | | | | | |

Downstream Tablet

| | | | | | |
|---|---|---|---|---|---|
| Mannitol | 224.6 | 224.6 | 224.6 | 224.6 | 224.6 |
| Croscarmellose | 50 | 50 | 50 | 0 | 50 |
| Sodium | | | | | |
| Sodium Starch | 0 | 0 | 0 | 50 | 50 |
| Glycolate | | | | | |
| Sodium Chloride | 50 | 0 | 0 | 0 | 0 |
| (granular) | | | | | |
| Sodium Chloride | 0 | 50 | 0 | 50 | 0 |
| (fine) | | | | | |
| Potassium Chloride | 0 | 0 | 50 | 0 | 0 |
| (fine) | | | | | |
| Colloidal Silicon | 1.25 | 1.25 | 1.25 | 1.25 | 1.25 |
| Dioxide | | | | | |
| Magnesium Stearate | 7.5 | 7.5 | 7.5 | 7.5 | 7.5 |
| (non-bovine) | | | | | |
| Total | 500 | 500 | 500 | 500 | 500 |
| Disintegration Time | 13:23 | 5:56 | 5:33 | 23:55 | 25:50 |
| (mm:ss) | | | | | |
| Compression Force | 10.1 | 10.1 | 10.1 | 11.4 | 10.1 |
| using 16/32″ RSC | | | | | |
| (Target kN) | | | | | |
| Hardness (kP) | 13.9 | 14.7 | 15.3 | 13.7 | 14.9 |
| Calculated Tensile | 1.92 | 2.04 | 2.10 | 1.97 | 1.96 |
| Strength (MPa) | | | | | |

It was surprisingly found that combinations of croscarmellose sodium in combination with NaCl or KCl provide an observed enhanced disintegration time.

### Example 4

### Copovidone-SLS Formulations—High Drug Loading

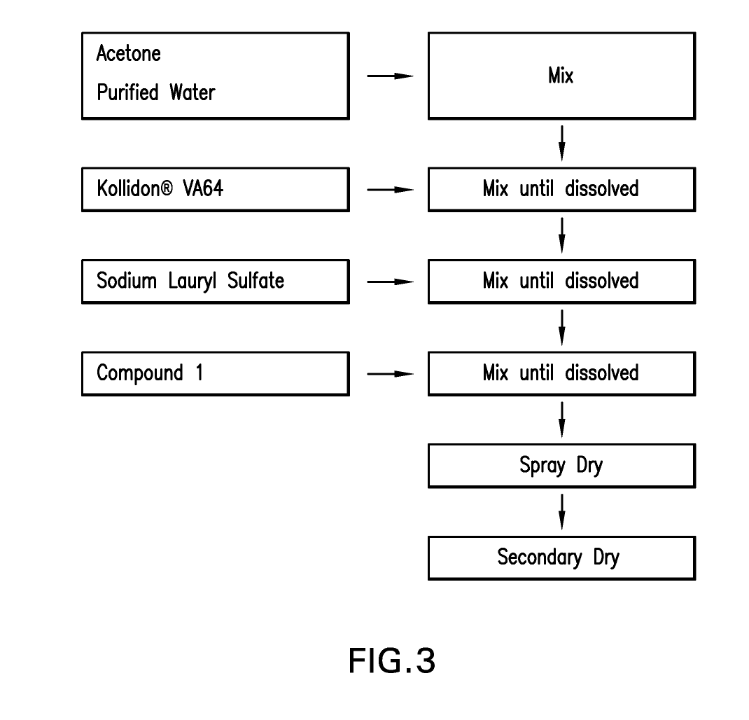

A formulation (Formulation 10) of Compound 1, SLS, and copovidone was prepared as described in Table 8 and FIG. 3 using a 90/10 acetone/water solution as the spray drying solvent. The concentration of Compound 1 in the dry spray dried intermediate of Formulation 10 was 40% w/w in comparison with the 30% w/w spray dried intermediate used in Formulation 4. The three solid components of the spray drying solution were incorporated into the solution at 14% w/w. A Niro PSD-1 spray dryer with a pressure nozzle was used to produce the spray dried particles. Heated nitrogen was supplied to the spray dryer at an inlet temperature sufficient to maintain a 53° C. outlet temperature and a gas flow rate of 1805 g/min. The spray drying solution flow rate was 140-170 g/min which required a nozzle pressure of approximately 150-170 PSI.

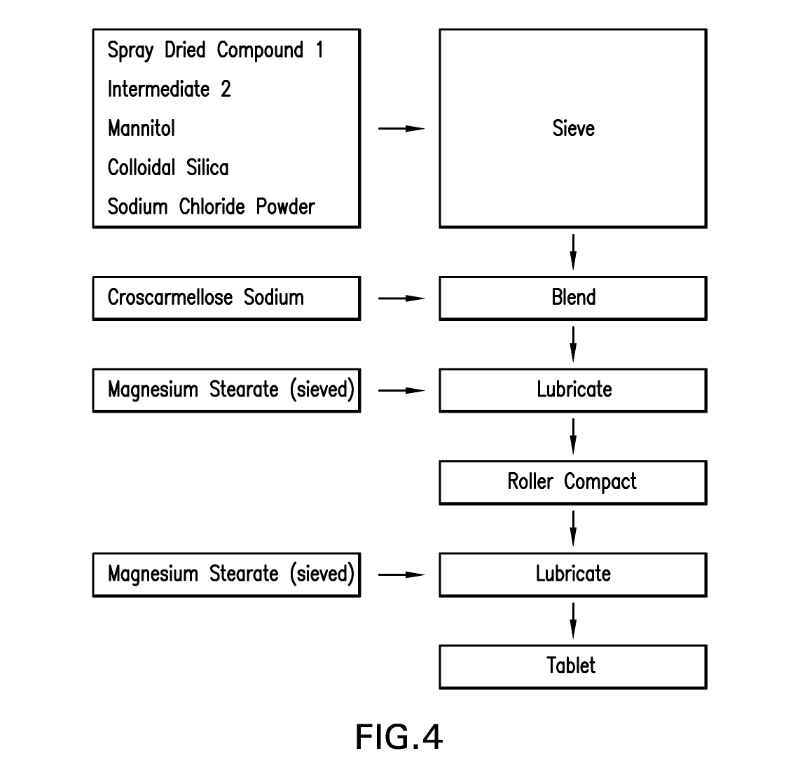

The dried spray dried intermediate (screen through No. 30 mesh) was blended with the “downstream tablet” components listed in Table 8 (screen through No. 30 mesh, except croscarmellose sodium), except the magnesium stearate, using a PK Blender for 10 minutes at 25 RPM in a process as described in FIG. 4. One third of the magnesium stearate (screen through No. 60 mesh) was added to the blender and the mixture was lubricated for 5 minutes at 25 RPM. The blend was then granulated into ribbons using an Alexanderwerk WP 120 Roller Compactor with a 25 mm knurled roll operating at a roll pressure of 28 bar with a roll gap of 2.0 mm. The ribbons were subsequently milled using the rotary fine granulator equipped with 2.0 mm and 1.0 mm size CONIDUR® screens. The granules were then blended with the remaining magnesium stearate (screened through No. 60 mesh) in the PK Blender for 5 minutes at 25 RPM. The lubricated granules were then compressed on a rotary tablet press (Korsch XL100) to a 1333 mg image tablet using 2 tablet stations with size 10.3 mm x 21.2 mm modified oval tooling. The hardness of the tablets was measured to be between 24 to 37 kiloponds (kp=1 kgf).

TABLE 8
Composition of Formulation 10

| | |
|---|---|
| | Amount |
| Components | (mg/tablet) |

Spray Dried Intermediate

| | |
|---|---|
| Compound 1 (free acid) | 200.0 |
| Polyvinylpyrolidone/Vinyl Acetate Copolymer | 275.0 |
| (Kollidon VA64) | |
| Sodium Lauryl Sulfate | 25.00 |

Downstream Tablet

| | |
|---|---|
| Mannitol | 543.4 |
| Croscarmellose Sodium | 133.3 |
| Sodium Chloride | 133.3 |
| Colloidal Silicon Dioxide | 3.333 |
| Magnesium Stearate (non-bovine) | 20.00 |
| Total | 1333 |

The absorption of Compound 1 following oral administration of Formulations 4 and 10 was evaluated in a human biocomparison study in the fasted state at a dose of 600 mg (Table 9). Increasing the concentration of Compound 1 in the spray dried intermediate from 30 to 40% results in a dramatic and unexpected decrease in absorption of Compound 1.

TABLE 9
Summary of Human Pharmacokinetic Results at 600 mg Dose

| Comparison | AUC_{0-∞}^{\|\|} | Cₘₐₓ^{\|\|} | C₂₄^{\|\|} |
|---|---|---|---|
| Formulation 10/ | 0.56 | 0.52 (0.39, 0.70) | 0.85 (0.76, 0.94) |
| Formulation 4 | (0.45, 0.70) | | |

^{||}GMR (90% CI).
GMR = Geometric least-squares mean ratio between formulations;
CI = Confidence interval.

### Example 5

### Copovidone-TPGS Formulation

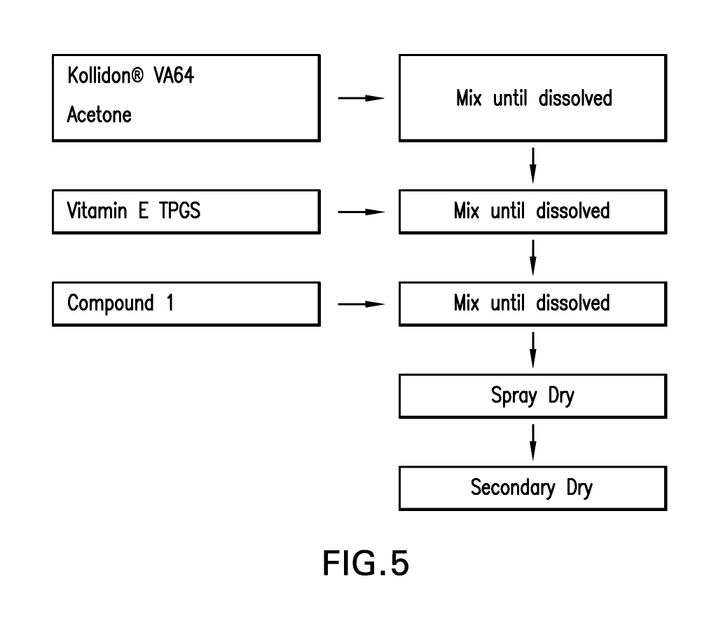

A formulation (Formulation 11) of Compound 1, Vitamin E TPGS, and copovidone was prepared as described by Table 10 and FIG. 5 using acetone as the spray drying solvent. The concentration of Compound 1 in the dry spray dried intermediate of Formulation 11 was 30% w/w in comparison with the 30% w/w spray dried intermediate used in Formulation 4. The three solid components of the spray drying solution were incorporated into the solution at 20% w/w. A Niro PSD-1 spray dryer with a pressure nozzle was used to produce the spray dried particles. Heated nitrogen was supplied to the spray dryer at an inlet temperature sufficient to maintain a 30° C. outlet temperature and a gas flow rate of 1850 g/min. The spray drying solution flow rate was 140-170 g/min which required a nozzle pressure of approximately 200-400 PSI.

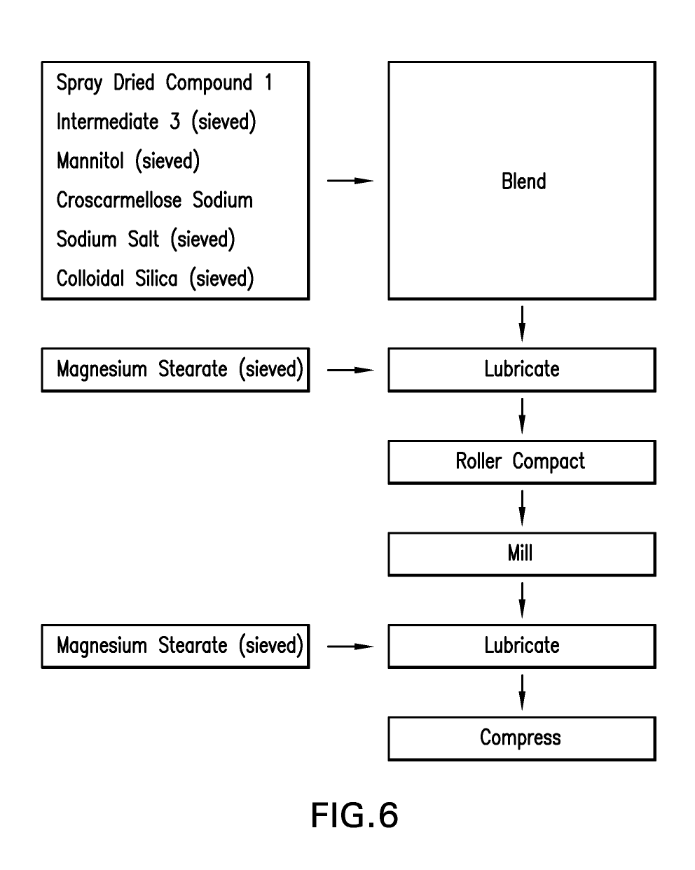

The dried spray dried intermediate (screen through No. 30 mesh) was blended with the “downstream tablet” components listed in Table 10 (screen through No. 30 mesh, except croscarmellose sodium), except the magnesium stearate, using a rotary blender for 10 minutes at 25 RPM in a process as described in FIG. 6. One third of the magnesium stearate (screen through No. 60 mesh) was added to the blender and the mixture was lubricated for 5 minutes at 25 RPM. The blend was then granulated into ribbons using an Alexanderwerk WP 120 Roller Compactor with a 25 mm knurled roll operating at a roll pressure of 19 bar with a roll gap of 2.0 mm. The ribbons were subsequently milled using the rotary fine granulator equipped with 2.0 mm and 1.0 mm size CONIDUR® screens. The granules were then blended with the remaining magnesium stearate (screened through No. 60 mesh) in the rotary blender for 5 minutes at 25 RPM. The lubricated granules were then compressed on a rotary tablet press (Piccola) to a 1000 mg image tablet using 2 tablet stations with size 9.74 mm×19.05 mm caplet tooling. The hardness of the tablets was measured to be between 14 to 24 kiloponds (kp=1 kgf).

TABLE 10
Composition of Formulation 11

| | |
|---|---|
| | Amount |
| Components | (mg/tablet) |

Spray Dried Intermediate

| | |
|---|---|
| Compound 1 (free acid) | 100.0 |
| Polyvinylpyrolidone/Vinyl Acetate Copolymer | 216.7.0 |
| (KOLLIDON ® VA64) | |
| Vitamin E TPGS | 16.67 |

Downstream Tablet

| | |
|---|---|
| Mannitol | 449.2 |
| Croscarmellose Sodium | 100.0 |
| Sodium Chloride | 100.0 |
| Colloidal Silicon Dioxide | 2.500 |
| Magnesium Stearate (non-bovine) | 15.00 |
| Total | 1000 |

Concentrations of Compound 1 in dog plasma were quantified by LC-MS/MS analysis (See, e.g., Example 1). The results are shown in Table 11.

TABLE 11
Pharmacokinetic data for Compound 1 following oral administration
to male beagle dogs (fasted state; 100 mg; pentagastrin was
administered intramuscularly at a target dose level of 6 μg/kg,
0.05 mL/kg 30 ± 5 minutes prior to dosing)

| | AUC_{0-24 hr} | | | AUC_{0-24 hr} |
|---|---|---|---|---|
| Formulation | (μM * hr) | Cₘₐₓ (μM) | Tₘₐₓ (hr) | ratio |
| Formulation 4 | 80.7 ± 16.9 | 11.6 ± 1.99 | 2.0 (2.0-4.0) | — |
| Formulation 11 | 87.4 ± 10.7 | 11.5 ± 0.742 | 2.0 (2.0-4.0) | 1.08 |

This example illustrates successful absorption of Compound 1 in an animal model from an orally administered formulation based on a solid solution intermediate comprising Compound 1, copovidone, and vitamin E TPGS.

### Example 6

### Copovidone Only Formulation (No Surfactant)

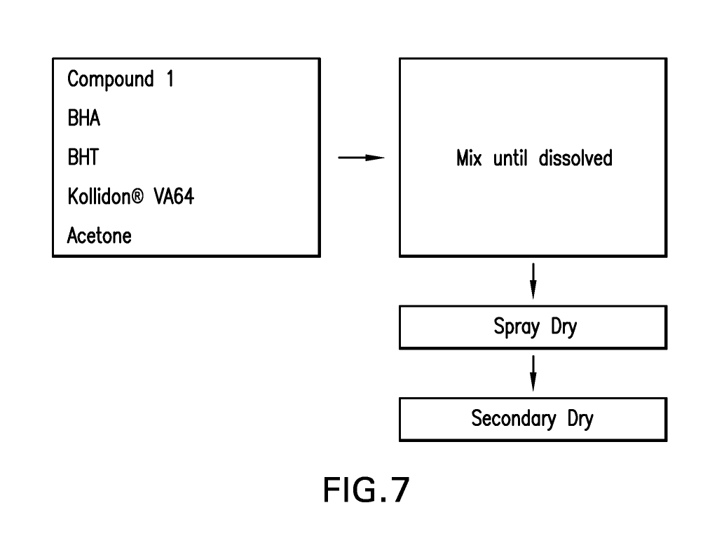

A formulation (Formulation 12) of Compound 1 and copovidone was prepared as described by Table 12 and FIG. 7 using a 90/10 acetone/water solution as the spray drying solvent. The concentration of Compound 1 in the dry spray dried intermediate of Formulation 12 was 33% w/w. The two solid components of the spray drying solution were incorporated into the solution at a total solids fraction of 20% w/w. A Niro PSD-1 spray dryer with a pressure nozzle was used to produce the spray dried particles. Heated nitrogen was supplied to the spray dryer at an inlet temperature sufficient to maintain a 41° C. outlet temperature and a gas flow rate of 1750 g/min. The spray drying solution flow rate was 220-280 g/min which required a nozzle pressure of approximately 120-220 PSI.

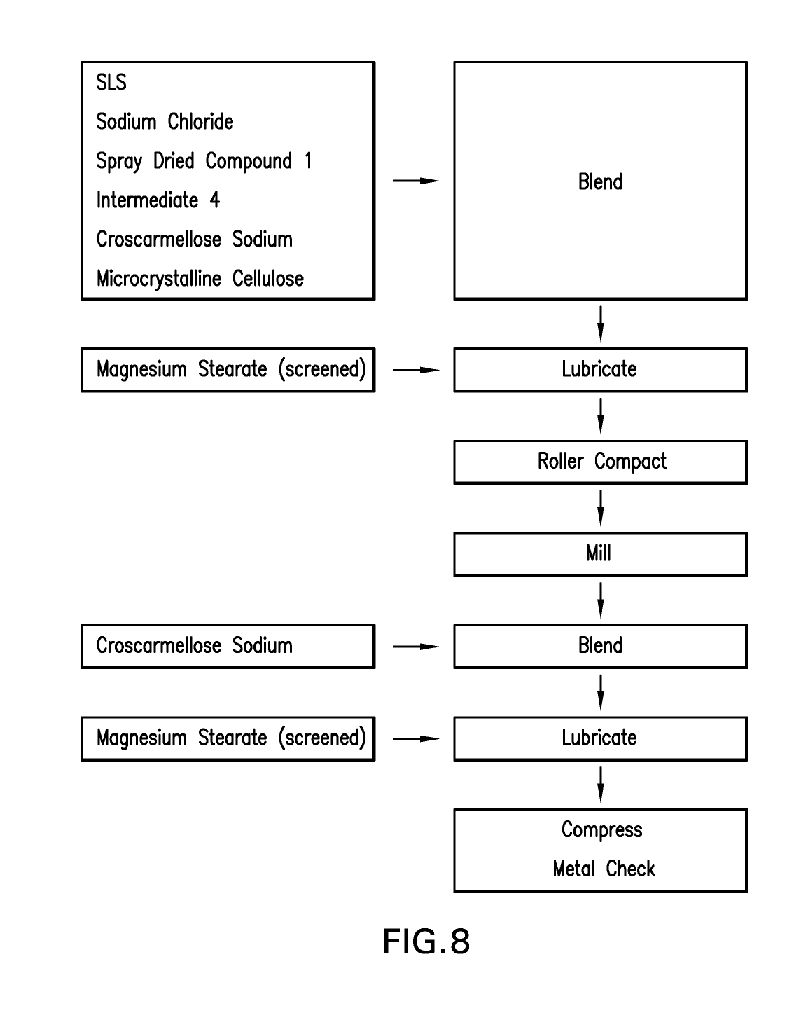

The dried spray dried intermediate (option to screen through No. 30 mesh) was blended with the “downstream tablet” components listed in Table 12, except the magnesium stearate and half of the croscarmellose sodium, using a 20 L Bin Blender for 10 minutes at 20 RPM in a process as described in FIG. 8. Half of the magnesium stearate (screen through No. 60 mesh) was added to the blender and the mixture was lubricated for 5 minutes at 20 RPM. The blend was then granulated into ribbons using an Alexanderwerk WP 120 Roller Compactor with a 40 mm knurled roll operating at a roll pressure of 2.0 MPa with a roll gap of 2.0 mm. The ribbons were subsequently milled using the rotary fine granulator equipped with 2.0 mm and 1.0 mm size CONIDUR® screens. The granules were then blended with the remaining croscarmellose sodium in the 20 L Bin Blender for 5 minutes at 20 RPM. The remaining magnesium stearate (screened through No. 60 mesh) was added and the mixture was lubricated in the 20 L Bin Blender for 5 minutes at 20 RPM. The lubricated granules were then compressed on a rotary tablet press to a 1000 mg image tablet using 2 tablet stations with size 7.94 mm×19.05 mm caplet tooling. The hardness of the tablets was measured to be between 18 to 24 kiloponds (kp=1 kgf).

The absorption of Compound 1 from Formulation 12 was evaluated by means of an oral administration study in fasted male beagle dogs and compared with Formulation 4 as a reference. See Table 13.

TABLE 12
Composition of Formulation 12

| | |
|---|---|
| | Amount |
| Components | (mg/tablet) |

Spray Dried Intermediate

| | |
|---|---|
| Compound 1 (free acid) | 200.0 |
| Polyvinylpyrolidone/Vinyl Acetate | 397.0 |
| Copolymer | |
| (KOLLIDON ® VA64) | |
| Butylated Hydroxyanisole | 1.500 |
| Butylated Hydroxy Toluene | 1.500 |

Downstream Tablet

| | |
|---|---|
| Microcrystalline Cellulose Avicel | 180.0 |
| Sodium Lauryl Sulfate | 50.00 |
| Croscarmellose Sodium | 60.00 |
| Sodium Chloride | 100.0 |
| Magnesium Stearate (non-bovine) | 10.00 |
| Total | 1000 |

TABLE 13
Summary of Human PK Results from Biocomparison Study
(600 mg dose; healthy subjects)

| Comparison | AUC_{0-∞}^{\|\|} | Cₘₐₓ^{\|\|} | C₂₄^{\|\|} |
|---|---|---|---|
| Formulation 12/ | 0.56 | 0.42 (0.29, 0.62) | 0.82 (0.73, 0.93) |
| Formulation 4 | (0.45, 0.71) | | |

^{||}GMR (90% CI).
GMR = Geometric least-squares mean ratio between formulations;
CI = Confidence interval.

The results shown in Table 13 illustrate the importance of including the surfactant in the solid solution itself (e.g., Formulation 4) instead of physically blending the surfactant with the spray dried solid solution intermediate particles (e.g., Formulation 12).

### Example 7

### HPMCAS

A formulation (Formulation 13) containing Compound 1 dispersed in hydroxypropyl methylcellulose acetyl succinate (HPMCAS) was prepared by spray drying Compound 1 and HPMCAS from 90:10 acetone:water. The concentration of Compound 1 in the dry spray dried intermediate of Formulation 13 was 40% w/w. The two solid components of the spray drying solution were incorporated into the solution at a total solids fraction of 14% w/w. A Niro PSD-1 spray dryer with a pressure nozzle was used to produce the spray dried particles. Heated nitrogen was supplied to the spray dryer at an inlet temperature sufficient to maintain a 51° C. outlet temperature and a gas flow rate of 1880 g/min. The spray drying solution flow rate was 220-280 g/min which required a nozzle pressure of approximately 150-180 PSI. 250 mg of the dispersion was weighed into a Pyrex bottle and 35 mL of SGF (Simulated Gastric Fluid) was added from a graduated cylinder. The suspension was agitated using a vibratory mixer. Settling of the formulation was noted over 2 hours. The suspensions were made on site and dosed quickly. During dosing the bottle for each individual animal was washed with some residual SGF to capture any residual particles in the bottle.

The male beagle dogs (weighing 11-14 kg) were selected and fasted overnight prior to dosing. Water was removed before dosing and was returned 1 hour after dosing. The dogs dosed with Formulation 4 received a 3.5 mL/kg water rinse via oral gavage following dosing. Food was returned at 4 hours after dosing. A 1 mL blood sample was drawn from the jugular vein into EDTA tubes at pre-dose and 0.25, 0.5, 1, 2, 4, 6, 8, and 24 hours after dosing. The plasma was separated by centrifugation (10 minutes at 2800 rpm) and kept frozen at −70° C. until analysis. Concentrations of Compound 1 in dog plasma were quantified by LC-MS/MS analysis. Area under the curve (AUC₀₋ₐₗₗ), observed maximum plasma concentration (Cₘₐₓ), time of Cₘₐₓ (Tₘₐₓ), means and SE were calculated using a linear trapezoidal, non-compartmental model of WinNonLin v5.01. Plasma profiles were generated using Excel® 2002 SP3 and WinNonLin v5.01.

TABLE 14
Pharmacokinetic data for Compound 1 following oral administration
to male beagle dogs (fasted state; 100 mg; pentagastrin was
administered intramuscularly at a target dose level of 6 μg/kg,
0.05 mL/kg 30 ± 5 minutes prior to dosing)

| | AUC_{0-24 hr} | | | AUC_{0-24 hr} |
|---|---|---|---|---|
| Formulation | (μM * hr) | Cₘₐₓ (μM) | Tₘₐₓ (hr) | ratio |
| Formulation 4 | 162 ± 26.2 | 18.5 ± 1.53 | 2.0 (2.0-2.0) | — |
| (n = 6) | | | | |
| Formulation 13 | 164 ± 35.7 | 20.2 ± 1.15 | 2.0 (1.0-4.0) | 1.01 |
| (n = 3) | | | | |

A suspension of Compound 1 dispersed in the cellulosic polymer, HPMCAS provided similar oral absorption of Compound 1 when compared to the tablet formulation 4.

While the foregoing specification teaches the principles of the present invention, with examples provided for the purpose of illustration, the practice of the invention encompasses all of the usual variations, adaptations and/or modifications that come within the scope of the following claims.

1. A composition comprising:
a) 1aR,5S,8S,10R,22aR)-5-tert-butyl-N-{(1R,2S)-1-[(cyclopropylsulfonyl)carbamoyl]-2-ethenylcyclopropyl}-14-methoxy-3,6-dioxo-1,1a,3,4,5,6,9,10,18,19,20,21,22,22a-tetradecahydro-8H-7,10-methanocyclopropa[18,19][1,10,3,6]dioxadiazacyclononadecino[11,12-b]quinoxaline-8-carboxamide (Compound 1), or a pharmaceutically acceptable salt thereof, in a concentration from 0.1% to 40% w/w; and
b) an absorption enhancing polymer in a concentration between 60% and 99.9% w/w.

2. The composition of claim 1, wherein Compound 1, or a pharmaceutically acceptable salt thereof, is present at a concentration from 5% to 35%.

3-5. (canceled)

6. The composition of any of claims 1-2, further comprising a surfactant in a concentration from 2% to 15%.

7. The composition of claim 6, wherein the absorption enhancing polymer is a cellulosic polymer or a copolymer of vinyl pyrrolidone and vinyl acetate.

8. The composition of claim 7, wherein the absorption enhancing polymer is a cellulosic polymer selected from HPMC or HPMCAS.

9. The composition of claim 7, wherein the copolymer of vinyl pyrrolidone and vinyl acetate is copovidone.

10. (canceled)

11. The composition of claim 6, wherein the surfactant is present at a concentration from 5% to 10% w/w and is selected from sodium lauryl sulfate (SLS) and D-α-tocopheryl polyethylene glycol 1000 succinate (vitamin E TPGS).

12. The composition of claim 1, wherein the composition is in the form of a particle.

13. A solid dispersion comprising particles of claim 12.

14. The solid dispersion of claim 13, wherein said dispersion is formed by spray drying or extruding the composition of claim 1.

15. The solid dispersion of claim 13, wherein said dispersion comprises particles wherein the particle further comprises SLS and is formed by spray drying in a mixed solvent system comprising a volatile solvent and a non-volatile solvent.

16. The solid dispersion of claim 15, wherein the volatile solvent is ethanol, methanol or acetone and the non-volatile solvent is water.

17-18. (canceled)

19. A blended material comprising the solid dispersion of claim 15, a salt, and a disintegrant present at a concentration of 5-20% w/w.

20. A pharmaceutical formulation comprising the solid dispersion of claim 15, a salt, and a disintegrant present at a concentration of 5-20% w/w.

21. The pharmaceutical formulation of claim 20 wherein the salt is selected from NaCl, KCl, CaCl₂ or a combination thereof.

22. (canceled)

23. The the pharmaceutical formulation of claim 20 wherein the disintegrant is croscarmellose sodium.

24-26. (canceled)

27. A process for preparing a solid pharmaceutical composition comprising the steps of:
a) dissolving the composition of a solvent system comprising a volatile solvent;
b) spray-drying the dissolved composition to form particles; and
c) compressing the particles into a tablet or filling into a capsule.

28. The process of claim 27 comprising the steps of:
a) dissolving the composition of claim 1 in a solvent system comprising a volatile solvent;
b) spray-drying the dissolved composition to form particles;
c) blending the particles with one or more of a diluent, disintegrant, salt, lubricant, and glidant;
d) subjecting the blended particles to roller compaction;
e) adding a lubricant; and
f) compressing the particles into a tablet or capsule.

29. (canceled)

30. The process of wherein the solvent system further comprises a non-volatile solvent.

31. (canceled)

32. The process of claim 27, wherein the solvent system is acetone:water (90:10).
